(19) United States
(12) Patent Application Publication
(10) Pub. No.: US 2016/0311948 A1
(43) Pub. Date: Oct. 27, 2016

(54) PROCESS FOR THE POLYMERIZATION OF DIACRYLATE-SUBSTITUTED HALOGENATED AROMATIC MONOMERS

(71) Applicant: BROMINE COMPOUNDS LTD., Beer-Sheva (IL)

(72) Inventors: Dov BERUBEN, Beer-Sheva (IL); Edith YA'ARI, Beer-Sheva (IL); Bertha CROITORU, Beer-Sheva (IL); Michael PELED, Beer-Sheva (IL)

(73) Assignee: BROMINE COMPOUNDS LTD., Beer-Sheva (IL)

(21) Appl. No.: 15/137,590

(22) Filed: Apr. 25, 2016

Related U.S. Application Data
(60) Provisional application No. 62/152,886, filed on Apr. 26, 2015.

(51) Int. Cl. C08F 122/18; C07C 67/62; C07C 67/10

(52) U.S. Cl. CPC C08F 122/18; C07C 67/10; C07C 67/62

(57) ABSTRACT

The invention relates to the free radical polymerization of tetrahalogen xylylene diacrylates monomers, either in a mixture of water-miscible aprotic solvent and water, or in a water-immiscible solvent, in the presence of a chain length modifier.

The invention relates to the polymerization of monomers which are tetrahalogen xylylene diacrylates, represented by Formula (I):

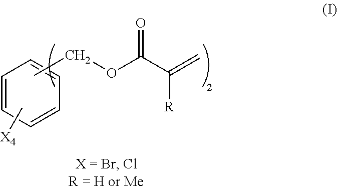

In Formula (I), the two —CH2-O—C(O)—C(R)═CH2 groups are attached to the benzene ring either in the ortho, meta or para orientation, and the four halogen atoms are independently selected from bromine and chlorine, i.e., mixed bromine and chlorine-containing compounds are also within the scope of Formula (I). Of particular interest, however, is the polymerization of tetrabrominated monomers, namely, tetrabromoxylylene bisacrylate (wherein in Formula (I), X is bromine and R is hydrogen) and tetrabromoxylylene bismethacrylate (wherein in Formula (I), X is bromine and R is methyl), especially the para compounds. As an alternative to the representation by means of Formula (I), the notation C6X4[-CH2-O—C(O)—C(R)═CH2]2 is sometimes used herein to indicate the monomers.

The synthesis of the monomers of Formula (I) and their polymerization is reported in U.S. Pat. No. 4,059,618 and U.S. Pat. No. 4,128,709. In the former patent, it is briefly stated that the polymers can be produced by means of solution polymerization in benzene.

However, the working examples of U.S. Pat. No. 4,059,618 deal only with the synthesis of the monomers. In the latter patent, solution polymerization of the monomers in methyl glycol in the presence of dibenzoyl peroxide or dicumyl peroxide as a free radical initiator is illustrated. It is noted that the monomers of Formula (I) are bifunctional, and hence their polymerization (either homopolymerization, or as cross-linking agents in copolymerization in the presence of other monomers), lead to the formation cross-linked polymers. Examples 29 and 31 of U.S. Pat. No. 4,128,709 specifically illustrate the homopolymerization of tetrabromo-p-xylylene bisacrylate in methyl glycol using dibenzoyl peroxide and dicumyl peroxide as polymerization initiators, respectively. The products of Examples 29 and 31 of U.S. Pat. No. 4,128,709 were identified as crosslinked, insoluble and infusible powdered polymers. Sieving was used for particle size analysis, indicating a wide range of particle size distribution of the so-formed homopolymers.

It has now been found that the width of particle size distribution of a polymer (especially homopolymer) derived from a monomer of Formula (I) is affected by the choice of the solvent employed in the solution polymerization of the monomer and also by the presence of a chain length modifier. A homopolymer consisting of a population of particles with narrow size distribution is formed on performing the free radical polymerization of the monomer of Formula (I) in solution, wherein the solvent is selected from the group consisting of:

(i) a mixture of at least one water-miscible aprotic solvent and water; and

(ii) a water-immiscible organic solvent.

Preferably, the polymerization reaction is carried out in the presence a chain length modifier.

Thus, the first variant of the invention is a free radical polymerization process, comprising polymerizing in solution a monomer of Formula (I), wherein the solvent comprises water-miscible aprotic solvent(s) and water, optionally in the presence a chain length modifier.

By the term “water-miscible solvent” is meant a solvent, which at 20□C can be added to water in an amount of at least 1 g per 100 g water with no phase separation. As noted above, according to the first variant of the invention, the reaction medium employed in the polymerization process consists of an organic solvent, which is water-miscible aprotic solvent, and water, preferably at weight ratios in the range from 10:1 to 1:1, more preferably from 5:1 to 1:1, e.g., from 4:1 to 2:1. The water-miscible aprotic solvent is preferably selected from the group consisting of linear, branched or cyclic ethers. Glycol ethers, namely, the group of solvents based on dialkyl ethers of ethylene glycol and diethylene glycol, are especially preferred. Exemplary solvents include the dimethyl ether of ethylene glycol [i.e., 1,2-dimethoxyethane (abbreviated EGDME); also known as glyme] and the dimethylether of diethylene glycol (also know as diglyme). Cyclic ethers such as tetrahydrofurane and 1,4-dioxane are also suitable for use in the invention. Other classes of water-miscible aprotic solvents which may be used are ketones and esters (e.g., ethyleneglycol diethylacetate). Preferred solvents have the formulas R1-OCH2CH2O—R2 or R1-OCH2CH2-O—CH2CH2O—R2 wherein R1 and R2 are independently selected alkyl groups (e.g., C1-C4 alkyl groups) or C(O)CH3.

The concentration of the monomer of Formula (I) is preferably from 1 to 75 wt %, more preferably from 10 to 40 wt % relative to the mixture of the organic solvent and water.

The polymerization reaction is accomplished in the presence of an initiator, namely, a free radical initiator which exhibits good solubility in the aqueous solvent mixtures. By the term “water-soluble initiator” is meant an initiator whose solubility in water at 20□C is at least 1 g/liter, e.g., at least 20 g/liter, and even more specifically, at least 200 g/liter. To this end, water soluble persulfate (—O3SOOSO3-) salts are especially preferred. Utilizable persulfates include potassium persulfate (27 g/l at 20° C.), sodium persulfate (238 g/l at 20° C.) and ammonium persulfate (228 g/l at 20° C.). Water soluble peroxides and hydroperoxides, as described in J. Org. Chem 60 (16), p. 5341-5345 (1995), can also be used. Water soluble free radical initiators operative in the present invention may be also selected from the class of Redox initiators, namely, a pair of initiators consisting of water soluble oxidant (e.g., a persulfate salt) in combination with a water soluble reductant (metabisulfite salt). Other useful thermal initiators may be selected from the group of water-soluble azo compounds, such as 2,2′-azobis-(2-amidinopropane.HC1). The initiator loading is preferably between 0.01 and 10% w/w based on the monomer, preferably about 0.5 to 5%, e.g. about 1%.

As noted above, the polymerization reaction of the monomer of Formula (I) is preferably carried out in the presence of a chain length modifier/regulator. According to the first variant of the invention, preferred chain length regulators are sulfur containing compounds, in particular thiol compounds (R1SH, wherein R1 indicates an organic moiety, e.g. R1 is an alkyl group composed of not less than 8 carbon atoms). For this purpose, mercapto-compounds, especially hydrocarbylmercaptans with 8-20 carbon atoms per molecule are preferred. Suitable examples include n-dodecyl mercaptan, n-octyl mercaptan, tertiary dodecyl mercaptan, tertiary nonyl mercaptan, tertiary hexadecyl mercaptan, tertiary octadecyl mercaptan, tertiary eicosyl mercaptan, secondary octyl mercaptan, secondary tridecyl mercaptan, cyclododecyl mercaptan, cyclododecadienyl mercaptan, aryl mercaptan like 1-naphthalene thiol etc. Mixtures of these compounds may also be used. The amount of the sulfur-containing chain length regulators may vary within a broad range, dependent on the solvent chosen and other variables related to the polymerization. 0.01 to 20 parts by weight of a chain length modifier, e.g., 0.01-10 (per 100 parts of the monomer) can be used.

The second variant of the invention is a free radical polymerization process, comprising polymerizing in solution a monomer of Formula (I), wherein the solvent is water-immiscible, preferably in the presence a chain length modifier.

By the term “water-immiscible solvent” is meant a solvent with solubility in water of less than 1 gr. per 100 gr. water, at room temperature (20° C.). The water-immiscible solvent is preferably selected from the group consisting of aromatic hydrocarbons, which may be halogenated or non-halogenated. For example, non-halogenated aromatic hydrocarbons such as alkyl-substituted benzenes, e.g., toluene, xylene and mesitylene can be used. An exemplary halogenated aromatic compound is chlorobenzene.

The concentration of the monomer is preferably from 1 to 70 wt %, more preferably from 10 to 30 wt % relative to the water-immiscible organic solvent.

The polymerization reaction according to the second variant is accomplished in the presence of a free radical initiator which is preferably selected from the group of peroxides or hydroperoxides (e.g., dicumyl peroxide) and azo compounds (e.g. those having cyano groups on the carbons attached to the azo linkage). The amount of the initiator is preferably between 0.01 and 10% w/w based on the monomer, preferably about 0.5 to 5%, e.g. about 1%.

The preferred chain length regulator used according to the second variant of the invention is α-methylstyrene. The amount of the chain length regulator may vary within a broad range, dependent on the solvent chosen and other variables related to the polymerization. 0.01 to 20 parts by weight of a chain length modifier, e.g., 0.01-10 (per 100 parts of the monomer of Formula (I)) can be used.

In general, either according to the first or second variants of the invention, the polymerization of the monomer of Formula (I) is carried out with stirring under heating, preferably at a temperature in the range from 70□C to the reflux temperature, e.g., in the range from 70□C to 120° C. It should be noted that the reaction is generally carried out under atmospheric pressure.

The polymerization is carried out by charging a reaction vessel with suitable amounts of the solvent, the monomer and the auxiliaries (the initiator and the chain length regulator), and maintaining the reaction mixture under heating (e.g., at the reflux temperature) for a sufficient time in order to allow the reaction to reach completion. The progress of the reaction can be monitored by high pressure liquid chromatography (HPLC) for the disappearance of the monomer.

Upon completion of the polymerization (either according to the first or second variants of the invention), the reaction mixture is cooled, whereby the product precipitates. The solid polymer is easily separable from the reaction mixture and can be recovered using conventional techniques, e.g., filtration or solvent evaporation etc. The solid polymer is washed with water and dried to constant weight.

The polymer formed by the free radical polymerization of the monomer of Formula (I) under the conditions set out above was subjected to particle size analysis. The results indicate that the width of the particle size distribution of the so-formed polymer is narrower than hitherto reported in the prior art. Particle size is an important factor in many industrial fields, including in the polymers industry, and a narrow particle size distribution is considered advantageous. It should be understood that the particle size distributions reported herein are assigned to the reaction-derived product, namely, the particle size distribution is determined by the polymerization reaction and not due to downstream operations which are known to affect particle size, such as milling etc.

The particle size analysis reported herein is based on laser diffraction (the instrument used is laser diffraction particle size analyzer Mastersize 3000, where the measurements were carried out using a dispersion of the particles in water). The particle size distribution is described by three values (based on a volume distribution): D50, which indicates the median; D90, which indicates the value where 90% of the distribution lies below this value; and D10, which indicates the value where 10% of the distribution lies below this value. The distribution width can also be described by calculating the span, defined by the following equation:

(D90−D10)/D50.

The invention provides poly (tetrabromoxylylene bisacrylate) with the following particle size distribution:

D10 not more than 15 μm, for example, 5≦D10≦12 μm.

D50 not more than 50 μm, for example, 18≦D50≦45 μm.

D90 not more than 140 μm, for example, 60≦D90≦130 μm.

The span of the distribution is from 2 to 3, preferably from 2.2 to 2.8, indicating the narrow size distribution of the polymers obtained. When the polymerization reaction takes place in the absence of a chain length modifier, then 100≧D90<130 μm; in the presence of a chain length regulator, 60≦D90<90 μm.

The invention also provides poly (tetrabromoxylylene bismethacrylate) with the following particle size distribution:

D10 not more than 10 μm, for example, 4≦D10≦8 μm.

D50 not more than 40 μm, for example, 15≦D50≦35 μm.

D90 not more than 110 μm, for example, 50≦D90≦100 μm.

The span of the distribution is from 2 to 3, preferably from 2.2 to 2.8. When the polymerization reaction takes place in the absence of a chain length modifier, then 80≦D90<100 μm; in the presence of a chain length regulator, 50≦D90<70 μm.

Polymers formed by the process of the invention, especially the poly (tetrabromoxylene bisacrylate) and poly (tetrabromoxylene bismethacrylate) with the particle size distribution described above, can be used to reduce the flammability of thermoplastics. The polymers can be added to the plastics in concentration of about 5 to 30 wt %.

The experimental results reported below indicate that the polymerization reaction in an aqueous medium appears to proceed more rapidly than the corresponding reaction in a non-aqueous medium (i.e., the first variant offers faster reaction rates). Furthermore, in the case of tetrabromoxylylene bismethacrylate, when operating under the conditions of the first variant, the addition of the modifier appears to have a stronger effect on the width of the particle size distribution of the polymerization product, than in the second variant. However, from the standpoint of industrial production, the use of water-immiscible organic solvent in the polymerization reaction still offers several advantages. It has been now found that the synthesis of the C6X4[-CH2-O—C(O)—C(R)═CH2]2 monomers can be accomplished in a water-immiscible organic solvent and water in the presence of a phase transfer catalyst, by the reaction of a starting material of the formula C6X4[-CH2Y]2, wherein X is as previously defined and Y is independently bromide or chloride, with an alkali metal salt of acrylic (or methacrylic) acid of the formula CH2=C(R)—COOM, wherein M indicates the alkali metal, which salt is preferably formed in-situ upon combining together in the reaction mixture acrylic (or methacrylic) acid and an alkaline base such as sodium hydroxide. Therefore, the synthesis of the monomer, taking place in the water-immiscible organic solvent, may be followed immediately by a polymerization reaction in the same solvent, thus obviating the need for monomer isolation prior to polymerization. However, the monomer may of course be isolated and purified prior to polymerization.

The chemical equation below illustrates the synthesis of the monomers:

C6X4[-CH2Y]2+2CH2=C(R)—COOM→C6X4[-CH2-O—C(O)—C(R)═CH2]2

The tetrahalogen xylylene dihalide {C6X4[-CH2Y]2}, acrylic (or methacrylic) acid and the phase transfer catalyst are mixed together at a temperature below 20° C., preferably below 10° C., in a water-immiscible solvent of choice (e.g., halogenated benzene such as chlorobenzene or alkyl-substituted benzene such as toluene). The aqueous alkali metal hydroxide, e.g., sodium hydroxide in an aqueous form, is then added slowly and the reaction mixture is stirred at the temperature set out above. On completion of the gradual addition of the base, the reaction mixture is heated, and maintained under heating and stirring until an almost complete conversion is observed. The diacrylate monomer {C6X4[-CH2-O—C(O)—C(R)═CH2]2} is then recovered from the reaction mixture using conventional methods.

As the preferred monomers of Formula (I) are tetrabromoxylylene bis(meth)acrylate, the preferred starting material for use in the synthesis of these monomers is to be chosen from tetrabromoxylylene dihalide, namely, C6Br4[-CH2Y]2, especially the dibromide (Y is Br) of Formula (II):

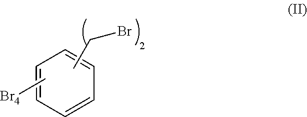

The phase transfer catalyst employed in the synthesis of the monomers can be selected from the group consisting of tetraalkylammonium halides. Tetrabutylammonium bromide is preferably used.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional application No. 62/152,886, filed Apr. 26, 2016 are incorporated by reference herein.

### EXAMPLES

### Example 1

Polymerization of tetrabromo-p-xylene bisacrylate

Reaction solvent: mixture of ethyleneglycol dimethylether and water

Initiator: ammonium persulafte

A 250 ml reactor equipped with a mechanical stirrer, a condenser and a thermometer was charged with 20 g of tetrabromo-p-xylene diacrylate, 120 ml of ethyleneglycol dimethylether and 40 ml of water. To this mixture was added 0.2 g of ammonium persulfate and the reaction mixture was heated to reflux. The reaction was monitored by HPLC to determine consumption of the monomer. After 4 h, the reaction was over. After cooling at room temperature, the solid is filtered, washed with water and dried to constant weight in an oven vacuum at 120° C.

The bromine content of the so-formed polymer, measured by the parrbomb method, is 56.6%. The particle size distribution is as follows: D(90)=129 μm; D(50)=43 μm; D(10)=10.2 μm.

### Example 2

Polymerization of tetrabromo-p-xylene bisacrylate

Reaction solvent: mixture of ethyleneglycol dimethylether and water

Initiator: ammonium persulafte

Chain length modifier: dodecanethiol

A 250 ml reactor equipped with a mechanical stirrer, a condenser and a thermometer was charged with 20 g of tetrabromo-p-xylene bisacrylate, 150 ml of ethyleneglycol dimethylether and 40 ml of water. To this mixture were added ammonium persulfate (0.2 g) and dodecanethiol (0.2 g), and the reaction mixture was heated to reflux. The reaction was monitored by HPLC to determine consumption of the monomer. After 4 h, the reaction was over. After cooling at room temperature, the solid is filtered, washed with water and dried to constant weight in an oven vacuum at 120° C.

The bromine content of the so-formed polymer, measured by the parrbomb method, is 54.6%. The particle size distribution is as follows: D(90)=84 μm; D(50)=33 μm; D(10)=8.6 μm.

### Example 3

Polymerization of tetrabromo-p-xylene bismethacrylate

Reaction solvent: mixture of ethyleneglycol dimethylether and water

Initiator: ammonium persulafte

The procedure of Example 1 was repeated, but this time the monomer which underwent polymerization was tetrabromo-p-xylene bismethacrylate.

The bromine content of the so-formed polymer, measured by the parrbomb method, is 53%. The particle size distribution is as follows: D(90)=96 μm; D(50)=34.3 μm; D(10)=6.6 μm

### Example 4

Polymerization of tetrabromo-p-xylene bismethacrylate

Reaction solvent: mixture of ethyleneglycol dimethylether and water

Initiator: ammonium persulafte

Chain length modifier: dodecanethiol

The procedure of Example 2 was repeated, but this time the monomer which underwent polymerization was tetrabromo-p-xylene bismethacrylate.

The bromine content of the so-formed polymer, measured by the parrbomb method, is 52.3%. The particle size distribution is as follows: D(90)=60 μm; D(50)=23.4 μm; D(10)=5.2 μm.

### Example 5

Polymerization of tetrabromo-p-xylene bisacrylate

Reaction solvent: toluene

Initiator: dicumylperoxide

A 250 ml reactor equipped with a mechanical stirrer, a condenser and a thermometer was charged with 20 g of tetrabromo-p-xylene bisacrylate and 80 ml of toluene. To this mixture was added 0.2 g of dicumylperoxide and the reaction mixture was heated to reflux. The progress of reaction was monitored by HPLC to determine consumption of the monomer. After 6 h, the reaction was over. After cooling at room temperature, the solid is filtered, washed with water and dried to constant weight in an oven vacuum at 120° C.

The bromine content of the so-formed polymer, measured by the parrbomb method, is 55.5%. The particle size distribution is as follows: D(90)=100 μm; D(50)=38 μm; D(10)=6.2 μm.

### Example 6

Polymerization of tetrabromo-p-xylene bisacrylate

Reaction solvent: toluene

Initiator: dicumylperoxide

Chain length regulator: α-methylstyrene

A 250 ml reactor equipped with a mechanical stirrer, a condenser and a thermometer was charged with 20 g of tetrabromo-p-xylene bisacrylate and 80 ml of toluene. To this mixture was added 0.2 g of dicumylperoxide and 0.2 g of α-methylstyrene and the reaction mixture was heated to reflux. The progress of reaction was monitored by HPLC to determine consumption of the monomer. After 22 h, the reaction was over. After cooling at room temperature, the solid is filtered, washed with water and dried to constant weight in an oven vacuum at 120° C.

The bromine content of the so-formed polymer, measured by the parrbomb method, is 54.3%. The particle size distribution is as follows: D(90)=60 μm; D(50)=20 μm; D(10)=5 μm.

### Example 7

Polymerization of tetrabromo-p-xylene bismethacrylate

Reaction solvent: toluene

Initiator: dicumylperoxide

The procedure of Example 5 was repeated, but this time the monomer which underwent polymerization was tetrabromo-p-xylene bismethacrylate.

The bromine content of the so-formed polymer, measured by the parrbomb method, is 52.4%. The particle size distribution is as follows: D(90)=55.4 μm; D(50)=19.3 μm; D(10)=4.8 μm

### Example 8

Polymerization of tetrabromo-p-xylylene bismethacrylate

Reaction solvent: toluene

Initiator: dicumylperoxide

Chain length regulator: α-methylstyrene

The procedure of Example 6 was repeated, but this time the monomer which underwent polymerization was tetrabromo-p-xylene bismethacrylate.

The bromine content of the so-formed polymer, measured by the parrbomb method, is 51.9%. The particle size distribution is as follows: D(90)=53.7 μm; D(50)=21.4 μm; D(10)5.3 μm.

### Example 9

Preparation of the Monomer tetrabromo-p-xylene bisacrylate

A 1 L reactor equipped with mechanical stirrer, condenser and thermometer was charged with chlorobenzene (600 ml) and acrylic acid (77.04 g, 1.07 mol). The reaction mixture is cooled to 10° C., followed by the addition of tetrabutylammonium bromide (0.5 g) and tetrabromo bis(bromomethyl)benzene (290 g, 0.5 mol). An aqueous sodium hydroxide solution, at a weight concentration of 48%, is slowly added (87.5 g, 1.05 mol) and the reaction mixture is heated to 95° C. and kept at that temperature for 5 h. The progress of the reaction is monitored by HPLC. On disappearance of the starting material, 300 ml of water is added and the chlorobenzene is distilled off. The slurry is cooled to room temperature and the solid is separated by filtration, giving 279 g (99% yield) of the entitled product. %Br (calculated 56.93%):57%.

### Example 10

Preparation of the Monomer tetrabromo-p-xylene bismethacrylate

The procedure of Example 9 was repeated, using methacrylic acid (92 g, 1.07 mol) instead of acrylic acid. 286 g (97% yield) of the entitled product is obtained. %Br (calculated 54.2%):54.6.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A free radical polymerization process, comprising polymerizing in solution in the presence of a free radical initiator, a monomer of Formula (I): 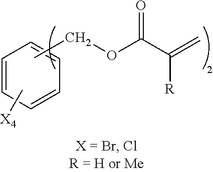
wherein the solvent comprises water-miscible aprotic solvent(s) and water.

2. A process according to claim 1, wherein the water-miscible aprotic solvent has the formula R1-OCH2CH2O—R2 or R1-OCH2CH2-O—CH2CH2O—R2, wherein R1 and R2 are independently selected from alkyl groups and C(O)CH3.

3. A process according to claim 2, wherein the solvent is selected from the group consisting of dimethyl ether of ethylene glycol and dimethyl ether of diethylene glycol.

4. A process according to claim 1, wherein the free radical initiator is a water-soluble persulfate salt.

5. A process according to claim 1, wherein the monomer is selected from the group consisting of tetrabromoxylene bisacrylate and tetrabromoxylene bismethacrylate.

6. A process according to any claim 1, wherein a chain length modifier is present in the reaction mixture.

7. A process according to claim 6, wherein the chain length modifier is thiol.

8. A process according to claim 7, wherein the thiol is dodecanethiol.

9. A free radical polymerization process, comprising polymerizing in solution a monomer of Formula (I): 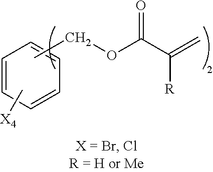
wherein the solvent is a water-immiscible solvent, in the presence of a free radical initiator and a chain length modifier.

10. A process according to claim 9, wherein the water-immiscible solvent is an aromatic hydrocarbon.

11. A process according to claim 10, wherein the aromatic hydrocarbon is selected from the group consisting of alkyl-substituted benzene and halogen-substituted benzene.

12. A process according to claim 9, wherein the free radical initiator is selected from the group of peroxides and hydroperoxides.

13. A process according to claim 9, wherein the chain length modifier is α-methylstyrene.

14. poly (tetrabromoxylene bisacrylate) having the following particle size distribution measured by laser diffraction:
D10 not more than 15 μm;
D50 not more than 50 μm; and
D90 not more than 140 μm.

15. poly (tetrabromoxylene bismethacrylate) having the following particle size distribution measured by laser diffraction:
D10 not more than 10 μm;
D50 not more than 40 μm; and
D90 not more than 110 μm.

16. A process for preparing compounds of Formula (I) 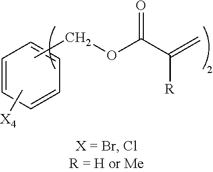
comprising the following reaction:
C6X4[-CH2Y]2+2CH2=C(R)—COOM→C6X4[-CH2-O—C(O)—C(R)═CH2]2
wherein X is independently bromine or chlorine, —CH2Y indicates a methyl side chain attached to the benzene ring, with Y independently being bromine or chlorine;
R is hydrogen or methyl; and
M is an alkali metal;
characterized in that the reaction takes place in a water-immiscible organic solvent and water in the presence of a phase transfer catalyst.
